(19) United States
(12) Patent Application Publication
(10) Pub. No.: US 2014/0069433 A1
(43) Pub. Date: Mar. 13, 2014

(54) LOW PROFILE MASK ATTACHMENT ELEMENT

(71) Applicants: Rick Wayne Walker, Stow, OH (US); Karl Herbert Beitzel, Canton, OH (US); Jason Anthony Belton, Norton, OH (US)

(72) Inventors: Rick Wayne Walker, Stow, OH (US); Karl Herbert Beitzel, Canton, OH (US); Jason Anthony Belton, Norton, OH (US)

(73) Assignee: KONINKLIJKE PHILIPS N.V., EINDHOVEN (NL)

(21) Appl. No.: 14/115,454

(22) PCT Filed: Apr. 30, 2012
(86) PCT No.: PCT/IB2012/052145

§ 371 Date: Nov. 4, 2013

Related U.S. Application Data
(60) Provisional application No. 61/485,800, filed on May 13, 2011.

(51) Int. Cl. A61M 16/06; A61M 16/08; A61M 16/00

(52) U.S. Cl. CPC A61M 16/0683; A61M 16/0057; A61M 16/0875; A61M 16/0816
USPC 128/206.21

(57) ABSTRACT

A patient interface device that includes a mask with a shell having a recess defined therein, thereon, or by a material coupled thereto and a headgear component for attaching the patient interface device to the head of a patient. The headgear component includes a strap and an attachment element provided between the mask and the strap, wherein the attachment element has a strap attachment portion coupled to the strap and a mask attachment portion disposed in the recess in a manner that selectively couples the headgear component to the mask.

### CROSS-REFERENCE TO RELATED APPLICATIONS

This patent application claims the priority benefit under 35 U.S.C. §119(e) of U.S. Provisional Application No. 61/485,800 filed on May 13, 2011, the contents of which are herein incorporated by reference.

### BACKGROUND OF THE INVENTION

1. Field of the Invention

The present invention relates to patient interface devices for transporting a gas to and/or from an airway of a user, and in particular, to a headgear component having a strap and an attachment element for coupling the headgear to a mask of a patient interface device.

2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation. It is also known to deliver continuous positive airway pressure (CPAP) or variable airway pressure, which varies with the patient's respiratory cycle, to treat a medical disorder, such as sleep apnea syndrome, in particular, obstructive sleep apnea (OSA), or congestive heart failure.

Non-invasive ventilation and pressure support therapies involve the placement of a patient interface device including a mask component on the face of a patient. The mask component may be, without limitation, a nasal mask that covers the patient's nose, a nasal pillow/cushion having nasal prongs that are received within the patient's nares, a nasal/oral mask that covers the nose and mouth, or a full face mask that covers the patient's face. The patient interface device interfaces the ventilator or pressure support device with the airway of the patient, so that a flow of breathing gas can be delivered from the pressure/flow generating device to the airway of the patient.

Furthermore, it is known to maintain such devices on the face of a patient by a headgear having one or more straps adapted to fit over/around the patient's head.

A number of smaller, “low profile” masks have been developed and commercially released within the past few years. A goal of such low profile masks is to make the masks less cumbersome and, thus, more comfortable for a patient to wear. An area of potential improvement in such low profile masks, as well as conventional masks, is in the mechanisms and elements used to secure such masks to the straps or headgear assemblies that are utilized in mounting such masks to the head of a patient.

### SUMMARY OF THE INVENTION

A patient interface device is provided that includes a mask having a shell with a recess defined therein, thereon, or by a material coupled thereto, and a headgear component for attaching the patient interface device to the head of a patient. The headgear component includes a strap and an attachment element provided between the mask and the strap, wherein the attachment element has a strap attachment portion coupled to the strap and a mask attachment portion coupled to the mask, and wherein the mask attachment portion is disposed in the recess in a manner that selectively couples the headgear component to the mask.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a schematic diagram of a system adapted to provide a regimen of respiratory therapy to a patient according to an exemplary embodiment of the invention;

FIG. 2A is an isometric view of an attachment mechanism according to one exemplary embodiment of the invention showing components in an uncoupled configuration;

FIG. 2B is an isometric view of the attachment mechanism of FIG. 2A showing components in a coupled configuration;

FIG. 2C is a detail view of a portion of one of the components shown in FIG. 2A;

FIG. 3A is an isometric view of an attachment mechanism according to another exemplary embodiment of the invention showing components in an uncoupled configuration;

FIG. 3B is an isometric view of the attachment mechanism of FIG. 3A showing components in a coupled configuration;

FIG. 4A is an isometric view of an attachment mechanism according to yet another exemplary embodiment of the invention showing components in an uncoupled configuration;

FIG. 4B is a cross-sectional view of the components of FIG. 4A arranged in a coupled position; and

FIGS. 5-8 are each top views of a mask attachment element according to alternative embodiments of the invention.

### DETAILED DESCRIPTION OF THE EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

As employed herein, the statement that two or more parts or components “engage” one another shall mean that the parts exert a force against one another either directly or through one or more intermediate parts or components.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

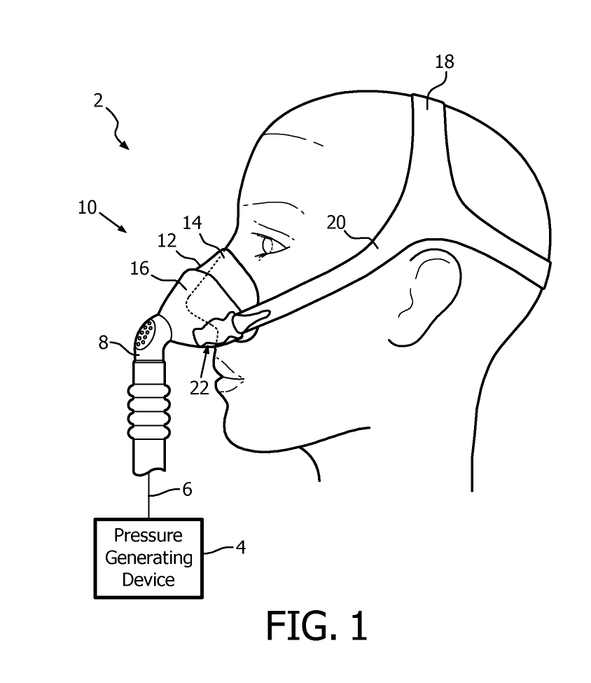

A system 2 adapted to provide a regimen of respiratory therapy to a patient according to an exemplary embodiment of the invention is generally shown in FIG. 1. System 2 includes a pressure generating device 4, a delivery conduit 6 coupled to an elbow connector 8, and a patient interface device 10. Pressure generating device 4 is structured to generate a flow of breathing gas and may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Philips Respironics of Murrysville, Pennsylvania), and auto-titration pressure support devices. Delivery conduit 6 is structured to communicate the flow of breathing gas from pressure generating device 4 to patient interface device 10 through elbow connector 8. Delivery conduit 6, elbow connector 8 and patient interface device 10 are often collectively referred to as a patient circuit.

Patient interface device 10 includes a mask 12, which in the exemplary embodiment is a nasal mask. However, any type of mask, such as a nasal/oral mask, a nasal pillow/cushion or a full face mask, which facilitates the delivery of the flow of breathing gas to the airway of a patient, may be used as mask 12 while remaining within the scope of the present invention.

Mask 12 includes a cushion 14 coupled to shell 16. Cushion 14 is made of a soft, flexible material, such as, without limitation, silicone, an appropriately soft thermoplastic elastomer, a closed cell foam, or any combination of such materials. In addition, shell 16 is made of a rigid or semi-rigid material such as, without limitation, polycarbonate or reinforced silicone. An opening (not numbered) in shell 16, to which elbow connector 8 is coupled, allows the flow of breathing gas from pressure generating device 4 to be communicated to an interior space defined by shell 16 and cushion 14, and then to the airway of a patient.

Patient interface device 10 also includes headgear component 18, which in the illustrated embodiment is a two-point headgear. Headgear component 18 includes first and second straps 20 each structured to be positioned on the side of the face of the patient above the patient's ear (only the left side strap 20 is shown). Headgear component 18 further includes first and second mask attachment elements 22 (one on each side the head of the patient; only the left side mask attachment element 22 is shown). As described in greater detail below, and as shown in FIG. 1, each mask attachment element 22 is provided to couple the end of one of the straps 20 to the respective side of mask 12.

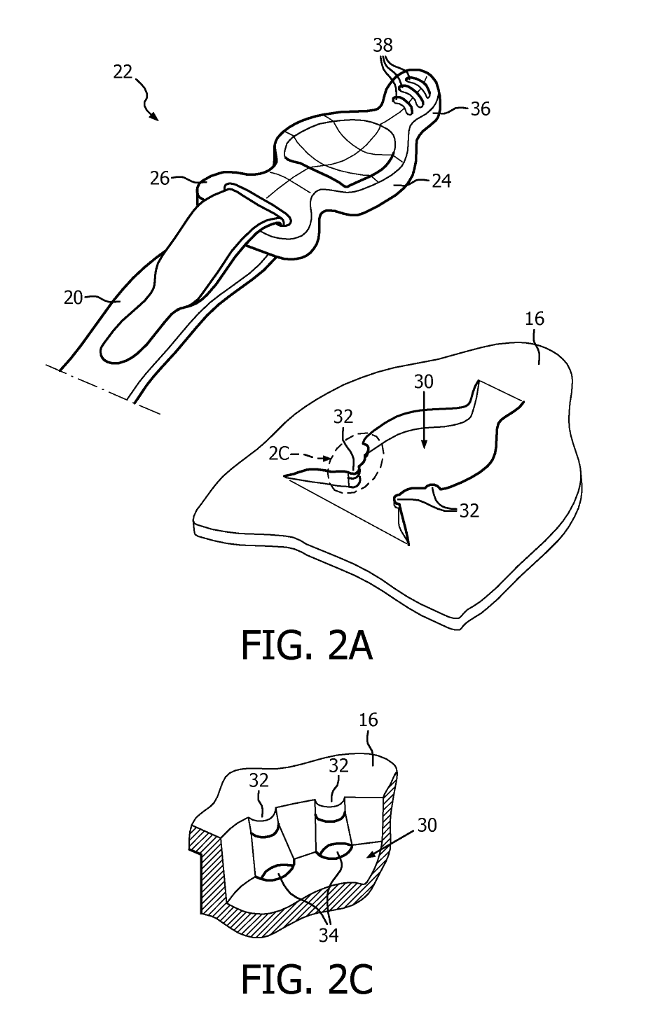

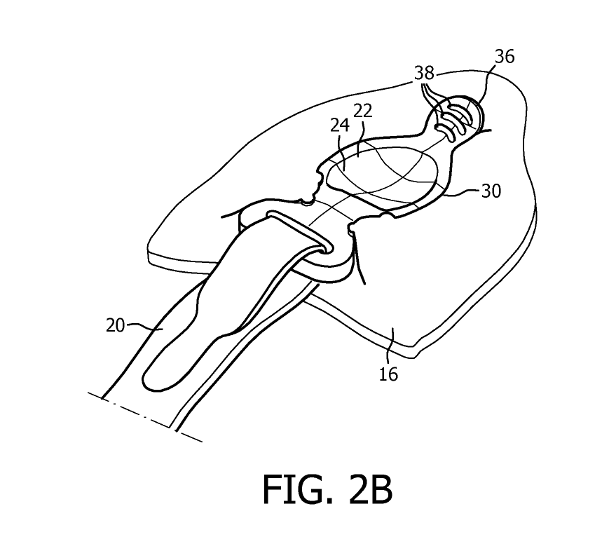

FIGS. 2A and 2B are isometric views of a portion of the exemplary embodiment of FIG. 1 showing mask attachment element 22 and portions of shell 16 of mask 12, and strap 20 of headgear 18. More particularly, FIG. 2A shows a configuration in which strap 20 is uncoupled from mask 16, and FIG. 2B shows a configuration in which strap 20 is coupled to mask 16. Referring to the uncoupled configuration of FIG. 2A, mask attachment element 22 generally includes a mask attachment portion 24 and a strap attachment portion 26.

Mask attachment portion 24 and strap attachment portion 26 may be integrally formed from a single material, such as shown in the embodiment of FIGS. 2A and 2B, or may be formed as initially separate components, from similar or different materials, which are then coupled together. In either case, mask attachment portion 24 is preferably made of a rigid or semi-rigid material, such as, without limitation, polycarbonate, reinforced silicone, high consistency silicone rubber (70 shore A and above), EVA copolymer, thermoplastic polyester elastomers (TPC-ET) (suitable examples of which are Hytrel® sold by DuPont, Riteflex® sold by Ticona), low-density polyethylene (LDPE), high-density polyethylene (HDPE), and polypropylene (PP).

In addition to being formed as a member separate from strap 20, as shown in FIGS. 2A and 2B, attachment element 22 may also be formed integrally with strap 20, thus providing a strap member that may be directly coupled to a mask without requiring an intermediate attachment element. In such instances, the integrated strap/attachment element is preferably formed from a silicone or other suitable material.

In order to couple strap 20 to shell 16 of mask 12, a recess 30 is provided on, in, or coupled to shell 16. In the example embodiment of FIGS. 2A, 2B and 2C, recess 30 is formed in a portion of shell 16 and is shaped to generally accommodate the entirety of mask attachment portion 24 of attachment element 22 therein, such as shown in the “coupled” view of FIG. 2B. Preferably, mask attachment portion 24 and recess 30 are cooperatively sized such that such elements tightly engage and are reliably coupled when positioned as shown in FIG. 2B.

In order to enhance the interaction between mask attachment portion 24 and recess 30 a number of further elements may be provided. For example, in order to create a more positive “coupled” fit, shell 16 may include a number of projections 32 which generally overhang or extend over a portion of recess 30. When mask attachment portion 24 is fully depressed into recess 30, such as shown in FIG. 2B, such projections 32 extend over respective portions (not numbered) of attachment portion 24, thus providing a further snap-like engagement between mask attachment portion 24 and recess 30.

As another example, such as shown in FIG. 2C, mask 16 may also include a number of apertures 34 disposed generally in recess 30 at or about the projections 32. Such apertures 34 may act to help provide ventilation to areas under mask 16 while also functioning to prevent the potential formation of air pockets between mask attachment portion 24 and mask 16. Such air pockets may form as a pocket of positive air pressure that would tend to resist compression, and thus act against depression of mask attachment element 24 down into recess 30, or alternately as a pocket of negative air pressure (vacuum) that would tend to act against removal of mask attachment portion 24 from recess 30.

To assist in removal of mask attachment portion 24 from recess 30, mask attachment portion 24 may also include one or more tabs, such as tab 36, which extend generally away from mask attachment portion 24. Preferably, such tab(s) include(s) one or more features 38 that increase the grip ability of the tab by a user of the patient interface device. Such features 38 may include, for example, without limitation, raised or depressed regions such as ridges, grooves, bumps, or depressions that may be integrally formed as a portion of, or formed separately and subsequently coupled to, tab 36. Although shown disposed generally opposite strap attachment portion 26, it is to be appreciated that tab 36 may be disposed in other portions about mask attachment portion 24 without varying from the scope of the present invention.

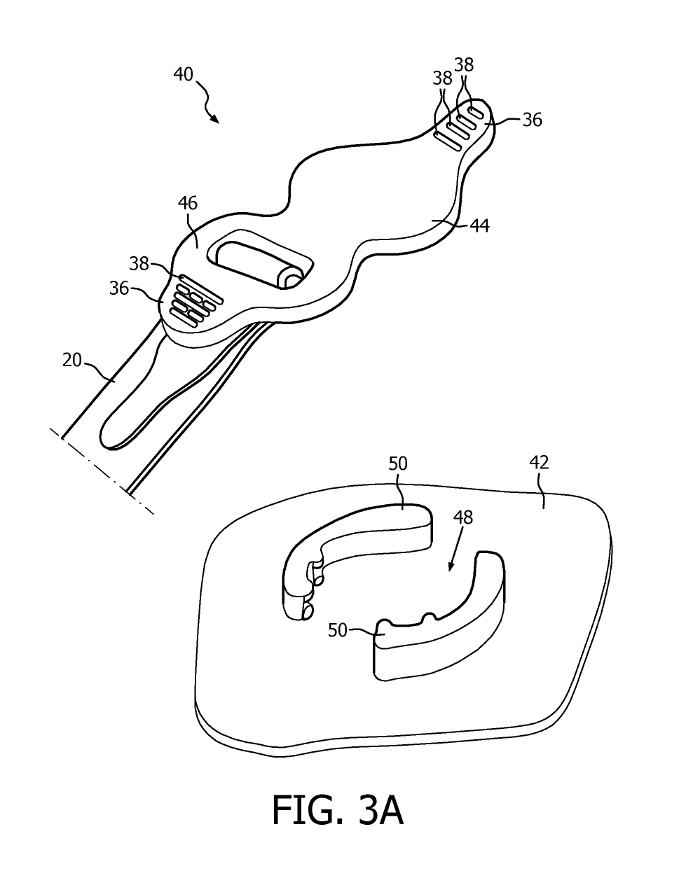

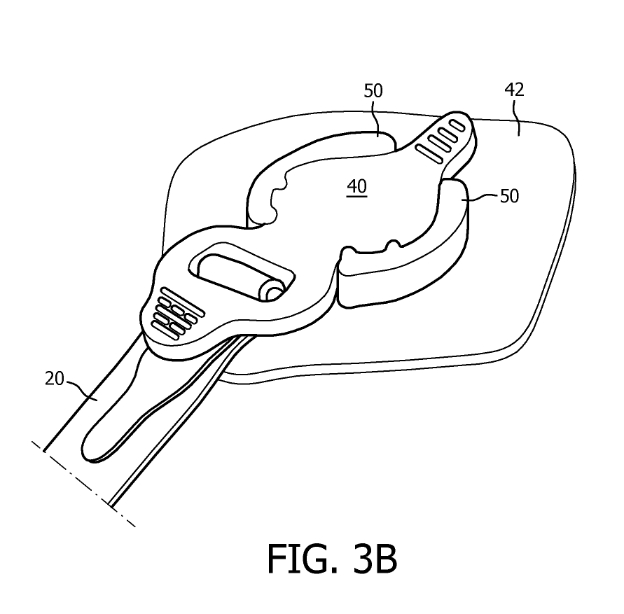

FIGS. 3A and 3B, respectively, show a mask attachment element 40 and a corresponding portion of a shell 42 of a mask (such as mask 12, FIG. 1) in uncoupled and coupled positions according to another exemplary embodiment of the present invention. Like the embodiment previously described in conjunction with FIGS. 2A-2C, mask attachment element 40 generally includes a mask attachment portion 44 and a strap attachment portion 46. Mask attachment portion 44 and strap attachment portion 46 may be integrally formed from a single material, such as shown in the embodiment of FIGS. 3A and 3B, or may be formed as initially separate components, from similar or different materials, which are then coupled together. In addition to being formed as a member separate from strap 20 of headgear 18, attachment element 40 may also be formed integrally with strap 20, thus providing a strap member that may be directly coupled to a mask without requiring an intermediate coupling member. In such instances, the integrated strap/attachment element is preferably formed from a silicone or other suitable material.

In order to couple strap 20 to shell 42, a recess 48 is provided on shell 42.

Recess 48 is generally defined by a number of raised wall sections 50 which protrude upward from a surface (not numbered) of shell 42. Although shown as being defined by two wall sections in FIGS. 3A and 3B, it is to be appreciated that a suitable recess 48 may also be defined by as few as one wall section or by a plurality of wall sections (i.e., more than two). Each wall section 50 may be formed as an integral portion of shell 42 or formed separately and coupled to shell 42. In either instance, wall sections 50 are preferably formed as rigid or semi-rigid structures. It is to be appreciated that aside from having a recess formed on a shell 42, the components shown in FIGS. 3A and 3B function in a like manner as those previously described in conjunction with FIGS. 2A-2C.

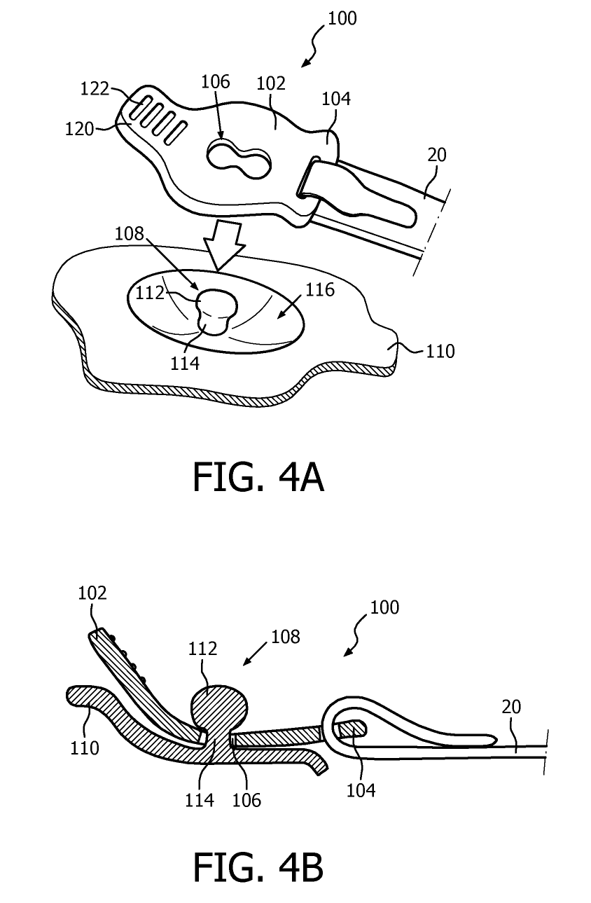

FIGS. 4A and 4B show another example attachment mechanism that may be employed independently or combined in whole or part with other embodiments of the present invention. Similar to the embodiments previously discussed, the attachment mechanism of FIGS. 4A and 4B includes an attachment element 100 which provides a coupling between a strap 20 and a mask, such as mask 12 of FIG. 1). Attachment element 100 includes a mask attachment portion 102 and a strap attachment portion 104 for coupling to a strap, such as strap 20 of patient interface device 10 of FIG. 1. Unlike the embodiments previously discussed, mask attachment portion 102 includes an aperture 106, preferably disposed generally at a mid-portion of attachment portion 102. Aperture 106 is sized to tightly engage a post 108 provided on a shell 110 of a mask (such as mask 12 of FIG. 1) in a generally snap-fit or similar manner. Post 108 preferably includes a bulbous portion 112 positioned at an upper portion a reduced neck portion 114 at a lower portion near shell 110. In the illustrated exemplary embodiment, aperture 106 and/or bulbous portion 112 is/are sized such that at least one dimension of aperture 106 is slightly smaller than bulbous portion 112. As shown in the cross-sectional view of FIG. 4B, such arrangement provides for attachment portion 102 to generally snap onto post 108, thus coupling strap 20 to shell 110.

In order to provide for a lower profile attachment and/or a even more secure attachment, post 108 is preferably provided within a recess 116 which may be formed as a generally concave-shaped depression in shell 110 (such as shown in FIGS. 4A and 4B) or formed in a more particular manner as previously discussed, on, in, or to a portion of a shell 110. Also, attachment element 100 preferably includes one or more tabs, such as tab 120 which extends generally away from mask attachment portion 102. Preferably, such tab(s) include(s) one or more features 122 that increase the grip-ability of the tab by a user of the patient interface device. As previously discussed, such features 122 may include, for example, without limitation, raised or depressed regions such as ridges, grooves, bumps, or depressions that may be integrally formed as a portion of, or formed separately and subsequently coupled to, tab 120. Although shown disposed generally opposite strap attachment portion 104, it is to be appreciated that tab 120 may be disposed in other portions about mask attachment portion 102 without varying from the scope of the present invention.

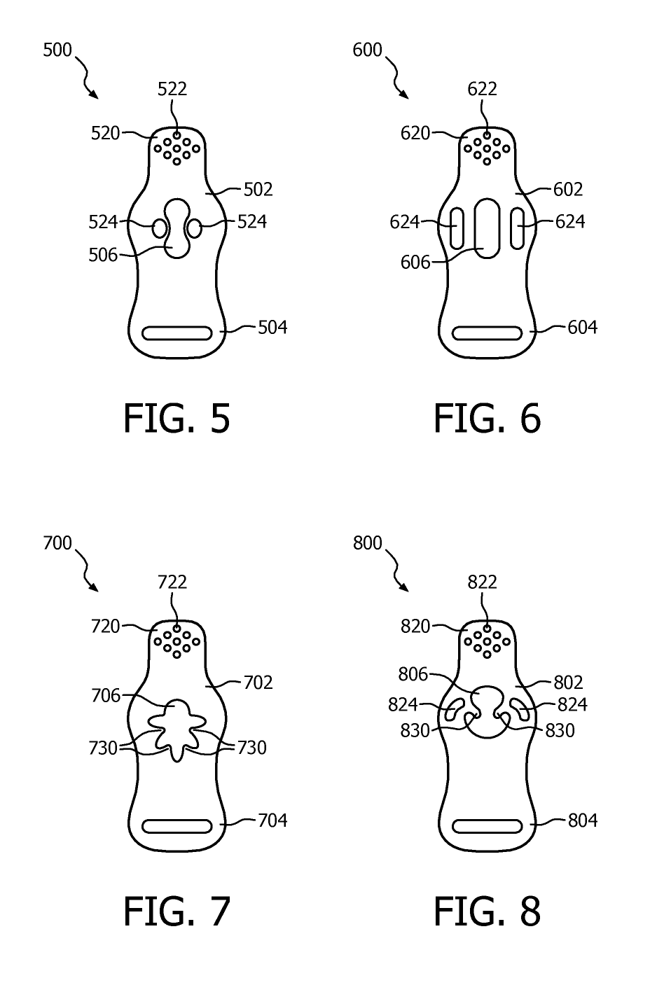

FIGS. 5-8 show examples of various attachment elements 500, 600, 700, 800 according to the present invention. It is to be appreciated that such examples are shown for example purposes only and are not intended to be limiting upon the scope of the present invention. Like attachment element 100, attachment elements 500, 600, 700, 800 include: mask attachment portions 502, 602, 702, 802, strap attachment portions 504, 604, 704, 804, apertures 506, 606, 706, 806 and tabs 520, 620, 720, 820 which preferably include features 522, 622, 722, 822 to increase grip-ability. As shown in the embodiments of FIGS. 5, 6 and 8, a number of second apertures 524, 624, 824 may be provided about apertures 506, 606, 806 to allow apertures 506, 606, 806 to flex slightly to allow such apertures to fit over a corresponding post (such as post 108 of FIGS. 4A and 4B) provided on a shell of a mask. As shown in the embodiments of FIGS. 7 and 8, attachment portion 702, 802 may include a number of projections 730, 830 which extend generally into apertures 706, 806. Such projections 730, 830 are preferably sized to engage (and flex as needed) a corresponding post provided on a shell of a mask.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination

Although example embodiments of the invention have been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A patient interface device comprising:
a mask including a shell having an inner side and an outer side, the shell having a recess defined in, on, or by a material coupled to the outer side, the recess opening outward from the shell, the recess having a depth; and
a headgear component for attaching the patient interface device to a head of a patient, the headgear component including a strap and an attachment element provided between the mask and the strap, the attachment element having a strap attachment portion coupled to the strap and a mask attachment portion cooperatively sized to the recess, the mask attachment portion having a thickness generally equal to the depth of the recess, the attachment portion being cooperatively engaged in the recess in a manner that selectively couples the headgear component to the mask,
wherein the mask attachment portion is adapted to be engaged in the recess by pressing the mask attachment portion inward toward the inner side of the shell and wherein the mask attachment portion is adapted to be disengaged from the recess by pulling the mask attachment portion outward away from the outer side.

2. The patient interface device of claim 1, wherein the shell comprises a number of projections extending over a portion of the recess, each of the number of projections being structured to selectively retain the mask attachment portion of the attachment element within the recess.

3. The patient interface device of claim 2, wherein the shell includes a number of apertures disposed at or about the number of projections.

4. The patient interface device of claim 1, wherein the attachment element is formed as an integral portion of the strap.

5. (canceled)

6. The patient interface device of claim 1, wherein the attachment element comprises a tab extending therefrom, the tab being adapted to be grasped by a user.

7. The patient interface device of claim 1, wherein the mask includes a post disposed in the recess and wherein the attachment element includes a first aperture formed therein that cooperatively engages the post.

8. The patient interface device of claim 7, wherein the attachment element includes a number of projections, each projection of the number of projections extending generally inward into the first aperture.

9. The patient interface device of claim 7, wherein the attachment element includes a number of second apertures disposed about the first aperture.

10. An attachment element for coupling a headgear component including a strap to a mask, the attachment element comprising:
a strap attachment portion adapted to be coupled to the strap; and
a mask attachment portion having a thickness adapted to be disposed within a cooperatively sized recess having a depth generally equal to the thickness of the mask attachment portion, the recess being formed in, on, or by a material coupled to, the mask, wherein the mask attachment portion is adapted to cooperatively engage the recess in a manner that selectively couples the attachment element to the mask when the mask attachment portion is pressed toward the mask and wherein the mask attachment portion is adapted to be disengaged from the recess when the mask attachment portion is pulled outward from the mask.

11. The attachment element of claim 10, wherein the attachment element is formed as an integral portion of the strap.

12. The attachment element of claim 11, wherein the attachment element and strap are formed from a silicone material.

13. The attachment element of claim 10, wherein the mask attachment portion includes a tab extending therefrom, the tab being adapted to be grasped by a user.

14. The attachment element of claim 10, wherein the mask attachment portion includes a first aperture formed therein, the first aperture being adapted to cooperatively engage a post disposed in the recess.

15. The attachment element of claim 14, further comprising a number of projections, each projection of the number of projections extending generally inward into the first aperture.

16. The attachment element of claim 14, further comprising a number of second apertures disposed about the first aperture.
